(19) United States
(12) Patent Application Publication
(10) Pub. No.: US 2024/0415816 A1
(43) Pub. Date: Dec. 19, 2024

(54) ADJUNCTIVE D-CYCLOSERINE AUGMENTATION OF TRANSCRANIAL MAGNETIC STIMULATION (TMS) THERAPY FOR OBSESSIVE COMPULSIVE DISORDER

(71) Applicant: MCGRX CORP., Calgary (CA)

(72) Inventor: ALEXANDER ROBERT ANGUS MCGIRR, Calgary (CA)

(21) Appl. No.: 18/712,677

(22) PCT Filed: Nov. 21, 2022
(86) PCT No.: PCT/CA2022/051708

§ 371 Date: May 22, 2024

Related U.S. Application Data
(60) Provisional application No. 63/282,297, filed on Nov. 23, 2021.

(51) Int. Cl. A61K 31/42; A61N 2/00; A61P 25/28

(52) U.S. Cl. CPC A61K 31/42; A61N 2/002; A61N 2/006; A61P 25/28

(57) ABSTRACT

There is provided adjunctive D-cycloserine (DCS) augmentation of transcranial magnetic stimulation (TMS) therapy for Obsessive Compulsive Disorder (OCD). There is also provided combination therapies and therapeutic methods that include intermittent or continuous theta burst stimulation, high- or low-frequency stimulation or combinations thereof, with D-cycloserine (DCS) to treat OCD in a patient and/or to improve and/or alleviate and/or reduce frequency of one or more symptoms of OCD, including cognitive function, and common comorbidities such as depression.

### FIELD OF THE INVENTION

This invention pertains generally to methods of treating Obsessive Compulsive Disorder (OCD) and, more particularly adjunctive D-cycloserine (DCS) augmentation of transcranial magnetic stimulation (TMS) therapy for Obsessive Compulsive Disorder (OCD) including theta-burst stimulation (TBS) or high- or low-frequency stimulation, or combinations thereof.

### BACKGROUND OF THE INVENTION

Obsessive Compulsive Disorder (OCD) is a significant source of global disability characterized by recurring and intrusive thoughts (obsessions) and ritualistic behaviors (compulsions). The prevalence in the general population is approximately 3%, ^{1, 2}. It ranks as one of the leading cause of global disability, particularly for those under 50 years of age³, and it has accounted for an increasing proportion of global disability adjusted life years in the last 25 years³. It is associated with significant direct and indirect cost to society⁴.

As 40-60% of patients do not experience significant improvement in their symptoms despite evidence based pharmacotherapy or psychological therapy ⁵, there exists a need for methods of treating OCD in patients where prior methods of treatment have been unsuccessful or not well tolerated.

In addition to the core symptoms of OCD, there are other important features of OCD that require consideration. An underrecognized association with OCD is suicidal ideation and behavior. In cross-sectional samples, as many as 28% of patients have current suicidal ideation⁶⁻⁸, and lifetime rates of suicide attempt are between 9-27%⁶⁻⁸. Cross-sectional samples belie the severity of risk, as prospective studies over follow-up periods as brief as 4 years report suicide attempts in 5% of patients and death by suicide in 0.9% of patients⁹.

Further, to these clinical features of OCD is cognitive dysfunction that has several consequences. Neuropsychological testing in OCD demonstrates impairments in several cognitive domains, in particular tests that relate to executive function, extra-dimensional shifting, working memory and spatial memory¹⁰⁻¹². These are generally regarded as poor prognosis factors¹³, and that these do not improve with existing treatments¹⁴. These represent a major challenge in the treatment needed in OCD.

Abnormalities in cortico-striatal-cortical circuits have been targeted using non-invasive brain stimulation as an alternative to conventional treatments for those who do not respond. In particular, non-invasive repetitive Transcranial Magnetic Stimulation (rTMS) of brain regions implicated in OCD has emerged as a treatment option for individuals who have not benefitted from or not tolerated pharmacotherapy. Rates of clinical response with rTMS are approximately 45% compared to 18% in sham stimulation in OCD¹⁵.

Accordingly, there exists a need for methods of enhancing rTMS for OCD as it also results in inadequate treatment outcomes.

Synaptic plasticity is thought to be the mechanism whereby rTMS results in improvements in OCD symptoms. In particular, pharmacological studies have shown that plasticity from the theta-burst protocol rTMS protocol TBS is N-methyl-D-aspartate receptor (NMDAR) dependent¹⁶. Although NDMAR agonists have been used as adjuncts to exposure and response prevention therapy, adequately powered trials have not found evidence for clinical benefit with this form of psychotherapy¹⁷⁻²⁰. This may be because the mechanisms of this treatment do not directly target synaptic plasticity and/or cortico-striatal-cortical circuitry. Pairing an adjunctive NMDAR agonist in combination with rTMS targeting cortico-striatal-cortical circuitry may improve the efficacy of rTMS in OCD.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide adjunctive D-cycloserine (DCS) augmentation of transcranial magnetic stimulation (TMS) therapy for Obsessive Compulsive Disorder. In accordance with an aspect of the invention, there is provided a method of treating Obsessive Compulsive Disorder (OCD) in a patient and/or improving and/or alleviating and/or reducing frequency of one or more symptoms of OCD, the method comprising: administering to the patient a low dose of D-cycloserine; and subjecting the patient to theta-burst stimulation (TBS), either intermittent or continuous, or high- or low-frequency stimulation, or combinations thereof.

In accordance with another aspect of the invention, there is provided a method of reducing suicidal ideation and suicide risk associated with Obsessive Compulsive Disorder (OCD), the method comprising: administering to the patient a low dose of D-cycloserine; and subjecting the patient to theta-burst stimulation (TBS), either intermittent or continuous, or high- or low-frequency stimulation, or combinations thereof.

In accordance with another aspect of the invention, there is provided a method of reducing cognitive dysfunction associated with Obsessive Compulsive Disorder (OCD), the method comprising: administering to the patient a low dose of D-cycloserine; and subjecting the patient to theta-burst stimulation (TBS), either intermittent or continuous, or high- or low-frequency stimulation, or combinations thereof.

In accordance with another aspect of the invention, there is provided a method of using D-cycloserine as a transcranial magnetic stimulation adjuvant, optionally theta-burst stimulation (TBS), or high- or low-frequency stimulation, or combinations thereof.

### BRIEF DESCRIPTION OF THE DRAWINGS

These and other features of the invention will become more apparent in the following detailed description in which reference is made to the appended drawings.

FIG. 1 shows motor cortex TMS plasticity data from a randomized placebo-controlled crossover trial (n=20) testing changes in stimulus response curve slopes, indicative of changes in neuronal recruitment, after a single and repeated spaced iTBS. This data indicates that in the iTBS+placebo condition changes in stimulus response curves are transient after a single iTBS and do not compound with repeated iTBS, whereas when delivered with DCS the changes in stimulus response curve slope are stable after a single iTBS and compound with spaced repeated iTBS. Slopes are normalized to the baseline stimulus response curve slope. Error bars indicate standard error of the mean.

FIG. 2 shows double-blind placebo- and sham-controlled randomized controlled trial data in patients with moderate severe OCD. This is a four group design, involving iTBS+Placebo, iTBS+DCS (100 mg), sham-iTBS+DCS (100 mg) and sham-iTBS+Placebo. As per pre-planned analyses, sham-iTBS+DCS and sham-iTBS+Placebo are pooled. The iTBS+DCS group has significantly greater improvements in YBOCS scores (OCD symptom severity scores) compared to both iTBS+Placebo and the pooled sham group. iTBS+DCS vs iTBS+Placebo * p<0.05 ** p<0.01; iTBS+DCS vs sham-iTBS-pooled ##p<0.01, ###p<0.001. Error bars indicate standard error of the mean.

FIG. 3 shows double-blind placebo- and sham-controlled randomized controlled trial data in patients with moderate severe OCD, as per FIG. 1. The iTBS+DCS group has significantly greater improvements as per the clinical global impression-improvement measure (CGI-1) compared to the Sham-iTBS group, whereas the iTBS+Placebo group did not. iTBS+DCS vs sham-iTBS-pooled. ** p<0.01. Error bars indicate standard error of the mean.

FIG. 4 shows double-blind placebo- and sham-controlled randomized controlled trial data in patients with moderate severe OCD, as per FIG. 1. The iTBS+DCS group has significantly greater improvements in depression (MADRS) scores compared to the Sham-iTBS group, whereas the iTBS+Placebo group did not. iTBS+DCS vs sham-iTBS-pooled. ** p<0.01. Error bars indicate standard error of the mean.

FIG. 5 shows double-blind placebo- and sham-controlled randomized controlled trial data in patients with moderate severe OCD, as per FIG. 1. The Wisconsin Card Sorting Task captures executive function changes that have been consistently implicated in OCD, namely the percentage of perseverative errors. The two-way repeated measures ANOVA indicates a significant group*time interaction (F(2,10)=12.97, p=0.0017) where the iTBS+DCS treated participants have significant reductions in the percentage of perseverative errors but not iTBS+Placebo or Sham-iTBS treated participants. Error bars indicate standard error of the mean.

### DETAILED DESCRIPTION OF THE INVENTION

Targeted neurostimulation treatments like TBS and other TMS protocols involve delivering trains of stimuli to drive activity dependent changes in the brain, referred to as synaptic plasticity. The efficacy of TBS is dependent on plasticity mechanisms and the N-Methyl-D-Aspartate receptor¹⁶. Yet, there are several lines of evidence indicating impaired TMS-associated plasticity in neuropsychiatric disorders²¹⁻²³, and OCD has been associated with consistent changes in cortical excitability as determined with TMS²⁴⁻²⁹. Responsiveness to TBS, or high- or low-frequency stimulation, or combinations thereof, may be improved by improving TMS-associated plasticity in patients with OCD.

One embodiment of the invention, therefore, provides an adjuvant therapy for TBS and other TMS protocols to increase TMS-associated plasticity. Optionally, increases in TMS-associated plasticity includes increased microstructural changes including neurite density, change in functional and branching and change in neuronal metabolite concentrations.

Accordingly, in one embodiment, the combination of TBS, or high- or low-frequency stimulation, or combinations thereof³⁰, with adjuvant therapies to improve TMS-associated plasticity is used in methods to treat OCD in a patient and/or to improve and/or alleviate and/or reduce frequency of one or more symptoms of OCD. The one or more symptoms of OCD include obsessions and compulsions.

In one embodiment, combination of TBS, or high- or low-frequency stimulation, or combinations thereof, with adjuvant therapies to improve TMS-associated plasticity is used in methods to treat common comorbidities of OCD, including depression, and/or to improve and/or alleviate and/or reduce frequency of one or more symptoms of these comorbid symptoms. The one or more symptoms of depression include feelings of sadness, tearfulness, hopelessness, short temper, irritation, loss of interest/lack of pleasure, memory loss, flat affect, sleep disorders, tiredness, reduced appetite and weight loss and/or feelings of worthlessness.

In one embodiment, combination of TBS, or high- or low-frequency stimulation, or combinations thereof, with adjuvant therapies to improve TMS-associated plasticity is used in methods to improve or restore cognitive flexibility, executive function, and/or working memory in a patient with OCD and/or improve or reverse or partially reverse cognitive impairments in a patient with OCD.

In one embodiment, combination of TBS, or high- or low-frequency stimulation, or combinations thereof, with adjuvant therapies to improve TMS-associated plasticity is used in methods to reduce risk of suicide or suicidal ideations in a patient with OCD. In some embodiments, the patient has a history of suicide attempts.

Appropriate TBS protocols are known in the art and include protocols for which there is sham-controlled evidence for efficacy. The iTBS stimulation frequency is constant with currently available technologies (3-pulse 50-Hz bursts at 5-Hz for 2-seconds trains, with trains every 10 seconds) or continuous for cTBS (3-pulse 50-Hz bursts at 5-Hz continuously). The total number of pulses per treatment and the treatment intensity may be varied. The number of pulses per treatment are between about 600 to about 1800. In some embodiments, each treatment includes 600 pulses. In other embodiments, each treatment includes 1200 pulses. In still other embodiments, each treatment includes 1800 pulses. The intensities can be varied to between about 80% to about 120% of motor threshold. In preferred embodiments, the intensity is between about 80% to 90% of motor threshold (rMT).

Appropriate high-frequency stimulation protocols are known in the art and include protocols for which there is sham-controlled evidence for efficacy. High frequency stimulation protocols involve trains of approximately 40 pulses at >5 Hz, with inter-train intervals of approximately 10-30 seconds. The intensities can be varied to between about 80% to about 120% of motor threshold. In preferred embodiments the intensity is between about 100% to 120% of resting motor threshold.

Appropriate high-frequency stimulation protocols are known in the art and include protocols for which there is sham-controlled evidence for efficacy. Low-frequency stimulation protocols involve trains of pulses at approximately 1 Hz, ranging from 300-2400 pulses. The intensities can be varied to between about 80% to about 120% of motor threshold. In preferred embodiments the intensity is between about 100% to 120% of resting motor threshold.

In some embodiments, one iTBS treatment occurs per a day. In other embodiments, multiple iTBS treatments occur daily, optionally between 2 and 10 treatments daily.

In some embodiments, iTBS, cTBS and other TMS protocols are delivered for between 1 and 52 weeks of treatment. In some embodiments, iTBS is continued to be provided after acute treatment courses to prevent relapse.

Synaptic plasticity associated with TBS, or high- or low-frequency stimulation, or combinations thereof, requires NMDAR signaling,^{16, 31} an ionotropic glutamate receptor that plays a major role in plasticity³². On binding glutamate these tetrameric receptors can initiate membrane depolarization, and through calcium signaling initiate intracellular messenger cascades, gene expression and protein synthesis to alter the strength of synaptic connections³³. Cellular surface expression of NMDAR is itself dynamically regulated and provides a secondary mechanism for long-term modulation of synaptic strength in a manner specific to acute and chronic stress^{34, 35}. Accordingly, in one embodiment of the invention, the adjuvant therapy for TBS, or high- or low-frequency stimulation, or combinations thereof, is to increase TMS-associated plasticity through NMDAR signaling.^{16, 31}

In one embodiment, combination of TBS, or high- or low-frequency stimulation, or combinations thereof, with NMDAR agonist or partial agonist is used in methods to reduce risk of suicide or suicidal ideations in a patient with OCD. In some embodiments, the patient has a history of suicide attempts.

In some embodiments of the invention, using NMDAR agonist or partial agonist as an adjuvant therapy for TBS, or high- or low-frequency stimulation, or combinations thereof, the NMDAR agonist or partial agonist is provided just before or concurrently with each treatment.

D-cycloserine (DCS) is a partial NMDAR agonist that preferentially binds to the glycine site of the NR2C NMDAR subunit^{36, 37}.

In one embodiment, combination of TBS, or high- or low-frequency stimulation, or combinations thereof, with D-cycloserine (DCS) is used in methods to treat OCD in a patient and/or to improve and/or alleviate and/or reduce frequency of one or more symptoms of OCD.

In one embodiment, combination of TBS, or high- or low-frequency stimulation, or combinations thereof, with D-cycloserine (DCS) is used in methods to improve overall cognitive function and/or improve/restore specific cognitive impairments in working memory or executive function/cognitive control in a patient with OCD.

In one embodiment, combination of TBS, or high- or low-frequency stimulation, or combinations thereof, with D-cycloserine (DCS) is used in methods to reduce risk of suicide or suicidal ideations in a patient with OCD. In some embodiments, the patient has a history of suicide attempts.

One embodiment of the invention, therefore, provides D-cycloserine (DCS) as an adjuvant therapy for TBS, or high- or low-frequency stimulation, or combinations thereof, to increase TMS-associated plasticity.

In embodiments of the invention, DCS is provided as a daily dose just prior to or concurrent with daily TBS, or high- or low-frequency stimulation, or combinations thereof, for 1-52 weeks. In some embodiments, DCS is provided <15 minutes, about 15, about 30 minutes, about 45 minutes, about 60 minutes, about 90 minutes, about 120 minutes, or about 360 minutes prior to iTBS treatment. Optionally, timing of TBS, or high- or low-frequency stimulation, or combinations thereof, treatment is dependent on plasma levels of DCS. Accordingly, in some embodiments, plasma levels of DCS must be above a minimal threshold prior to initiating iTBS or cTBS treatment. Optionally, the minimal threshold is 10 μg/mL.

In some embodiments, the methods of the invention comprise low dose D-cycloserine (DCS). Low dose D-cycloserine (DCS) includes doses ranging from about 1-250 mg of DCS. In preferred embodiments, the dose ranges from about 50 mg to about 150 mg of DCS. Appropriate doses of D-cycloserine may be based on patient's weight or resulting in a 1-30 μg/mL plasma concentration. Optionally, plasma concentration of D-cycloserine is assessed prior to the TBS, or high- or low-frequency stimulation, or combinations thereof, treatment.

In some embodiments, the D-cycloserine is provided as a pharmaceutically acceptable salt of D-cycloserine, a pharmaceutically acceptable ester of D-cycloserine, an alkylated D-cycloserine, or a pharmaceutically acceptable precursor of D-cycloserine.

In some embodiments, the D-cycloserine is provided as a pharmaceutical composition and may include pharmaceutically acceptable carriers or diluents.

The pharmaceutical compositions comprising D-cycloserine can be administered to the patient by any, or a combination, of several routes, such as oral, intravenous, trans-mucosal (e.g., nasal, vaginal, etc.), pulmonary, transdermal, ocular, buccal, sublingual, intraperitoneal, intrathecal or intramuscular. In some embodiments the pharmaceutical composition is formulated for rapid absorption and distribution.

Oral, sublingual, or buccal pharmaceutical formulations, such as tablets, capsule, sprinkle formulations, and oral suspensions, are preferred. In some embodiments, solid compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, lipids, alginic acid, or ingredients for controlled slow release. Disintegrators that can be used include, without limitation, micro-crystalline cellulose, corn starch, sodium starch glycolate and alginic acid. Tablet binders that may be used include, without limitation, acacia, methylcellulose, sodium carboxymethylcellulose, polyvinylpyrrolidone (Povidone), hydroxypropyl methylcellulose, sucrose, starch, and ethylcellulose.

### EXAMPLES

### Example 1: D-Cycloserine Stabilizes Motor Plasticity after Transcranial Magnetic TBS after a Single or Repeated Spaced Treatments

To assess if D-cycloserine, a NMDA-R partial agonist, can enhance TMS motor plasticity after a single and repeated spaced TBS treatments, a randomized double-blind placebo-controlled crossover study in healthy participants was completed.

Methods: A randomized double-blind placebo-controlled crossover study in healthy participants (n=20) was conducted (NCT05081986). Participants completed the study in two experimental sessions separated. In these experiments, the motor cortex was stimulated twice using TMS intermittent theta burst stimulation (iTBS) with a one-hour interval between interventions. In random order, this was done with placebo or D-cycloserine (100 mg) as adjuvant, and then the converse at least one week later.

The participants' surface anatomical markers were registered to a template magnetic resonance brain image using Neuronavigation software (ANT Neuro, Germany) to permit neuronavigation and reliable targeting throughout the experiment. Transcranial magnetic stimulation involved a MagPro X100 system with Cool-B70 figure-eight coil (MagVenture, Denmark). The iTBS protocol consisted of 20 trains of bursts of 3-pulses at 50 Hz, repeated at 5 Hz with an 8 second inter-train interval at 80% RMT.

Motor evoked potentials (MEPs) from the first-dorsal interosseous muscle were sampled to describe stimulus response curves using CED Signal (Cambridge, England) before and after iTBS. Stimulus response curves (SRC) were acquired by delivering TMS pulses at 0.25 Hz and randomly varying stimulus intensity between 100-150% resting motor threshold (RMT). For each stimulus intensity, a total of ten pulses were delivered. Stimulus response curves (SRC) were characterized at baseline, +30 minutes, +60 minutes after the first iTBS, and then +30 and +60 after the second iTBS.

EMG data were analyzed offline using MATLAB via custom written scripts as previously described¹²³. The peak-to-peak amplitude corrected to the mean value in the 100 ms window preceding the TMS pulse was isolated. EMG traces were individually inspected before inclusion in analyses. For analyses, we characterized the slope of the SRC sigmoidal function to determine change in recruitment curves. These were normalized to the baseline SRC slope for graphical representation.

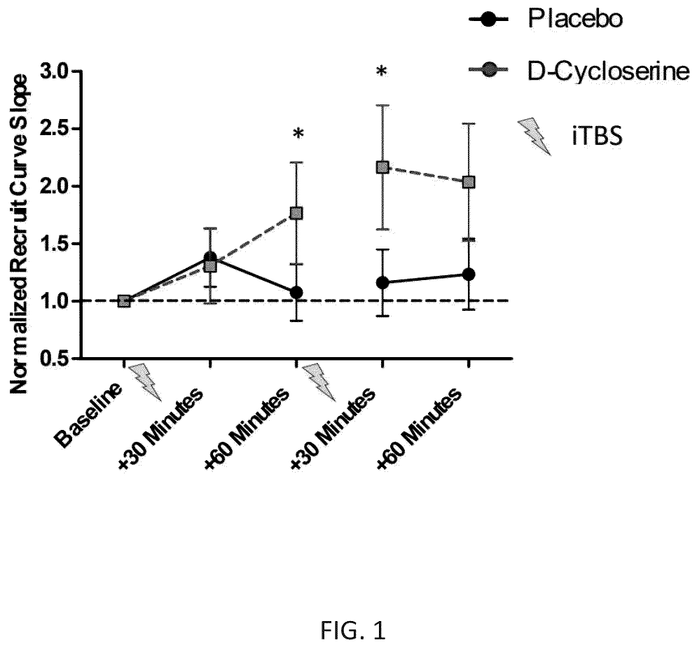

Results: Referring to FIG. 1, after the first iTBS in the placebo condition, the SRC transiently shifts +30 minutes before returning to baseline +60 minutes. In the DCS condition, the initial SRC shift at +30 minutes remains stable at +60 minutes. A second iTBS delivered 60 minutes after the first did not result in changes to the SRC in the placebo condition but resulted in a further shift in SRC slope in the DCS condition at +30 minutes that was sustained at +60 minutes.

Conclusion: NMDA-R agonism leads to persistent synaptic plasticity as measured by stimulus response curves after a single and repeated spaced iTBS interventions.

### Example 2: DCS in Combination with iTBS Enhances Clinical Outcomes in OCD

The efficacy of 100 mg DCS as an adjunctive strategy to iTBS was tested in a randomized double-blind 4-week study with both placebo- and sham-controlled arms, with follow-up at 1-month post-treatment. The trial was a double-blind randomized placebo- and sham-controlled trial follow-up at 1-month post-treatment, registered on clinicaltrials.gov and conducted according to Good Clinical Practice standards.

The reduction in OCD and depressive symptoms in iTBS+DCS participants from baseline to the completion of the double-blind treatment phase will be compared to iTBS+Placebo and Sham-iTBS treated participants.

Participants were randomized 2:2:1:1 to one of four conditions: 1) active-iTBS+DCS, 2) active-iTBS+Placebo, 3) sham-iTBS+DCS, or 4) sham-iTBS+Placebo. In this study, we utilized the MagPro X100 (MagVenture, Denmark) TMS device in combination with a Cool D-B80A/P coil capable of delivering sham and active stimulation. Participants received either daily iTBS+cycloserine, iTBS+placebo, sham-iTBS+cycloserine or sham-iTBS+placebo for 4 weeks (20 rTMS sessions) once daily on weekdays. Prior to the first treatment each subject's motor threshold (MT) will first be determined using an established method of neuronavigated TMS over the primary motor cortex. A short stimulation procedure was performed called motor threshold testing to determine the proper strength of the rTMS. The RMT was defined as the minimum stimulation intensity required to elicit an involuntary movement of the contralateral extensor hallicus longus in at least five out of ten attempts.

The iTBS protocol involves 50 Hz bursts, repeated at 5 Hz; 2 s on and 8 s off; 600 pulses per session delivered at 80% rMT³⁸. This is the stimulus intensity that was utilized in our pilot motor physiology data. The target will be the MPFC by advancing the coil 4 cm anterior to the extensor hallicus longus motor cortex representation.

DCS was placebo-control repackaged into 100 mg capsules. Capsules were ingested 2 hours before each treatment³⁹.

Participants received daily TMS in combination with the oral placebo-controlled oral compound (Monday to Friday) for four weeks.

Participants were assessed by a clinician at baseline, at 2 weeks and at 4 weeks. Self-reported measures were obtained weekly during the double-blind phase. Longer-term follow-up for participants treated with active-iTBS participants was scheduled for 1 month after treatment end. To mitigate unblinding sham-treated participants, participants who have not responded (≥30% improvement in the YBOCS) were be offered 4 weeks of open-label treatment.

The Primary Efficacy Outcome is change in clinician rated OCD symptoms using the YBOCS after 4 weeks of treatment. Secondary Efficacy Outcomes at 4 weeks include rates of clinical response (≥30% reduction in YBOCS score), Clinical Global Impression, and improvements in depressive symptoms MADRS, as well as effects over the 1-month follow-up phase.

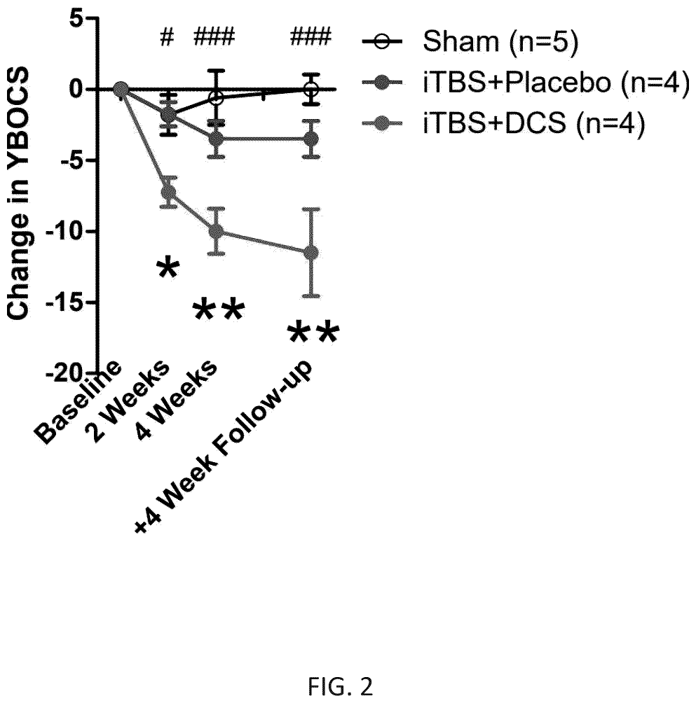

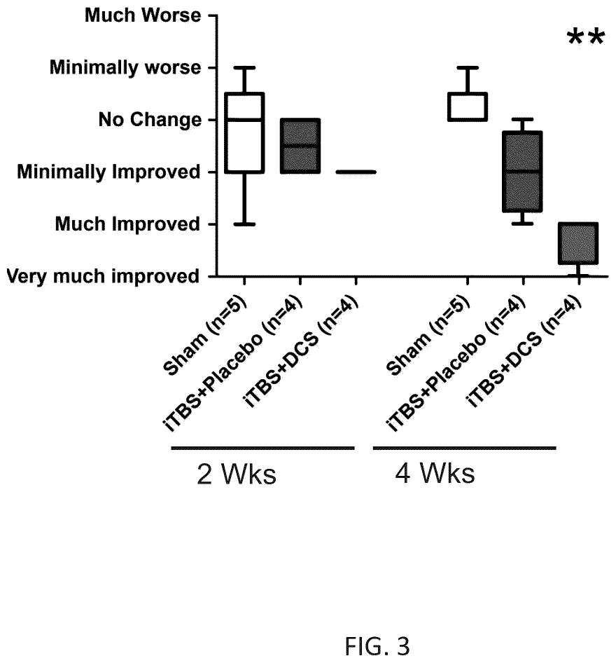

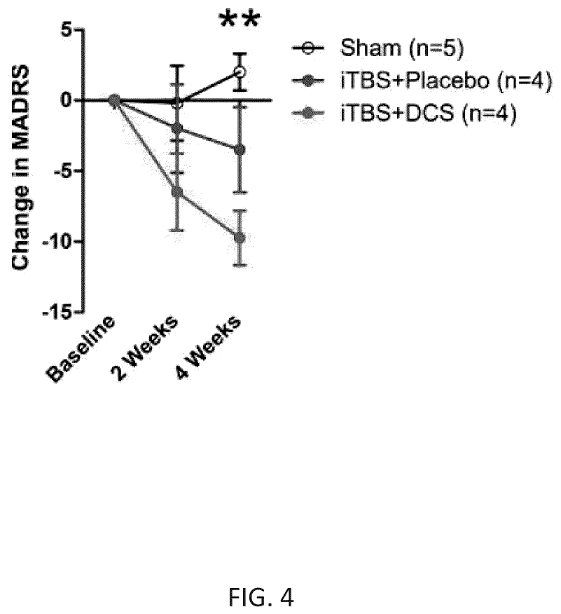

Results: Referring to FIG. 2, there was statistical separation with clinically meaningful differences on the YBOCS measure of OCD symptoms favoring the iTBS+DCS group compared to both the iTBS+Placebo group and the Sham-iTBS group. These effects were seen during the acute treatment period and were sustained over one month. Referring to FIG. 3, these improvements were paralleled by greater global improvement in the iTBS+DCS group, as measured by the clinician rated Clinical Global Impression scale. Referring to FIG. 4, depressive symptoms as measured by the MADRS improved significantly more in the iTBS+DCS group.

Conclusions: Adjunctive DCS with iTBS improves symptoms of OCD, depressive symptoms, and global function relative to iTBS+Placebo and Sham-iTBS.

### Example 3: DCS in Combination with iTBS Reduces OCD-Related Cognitive Impairments

The efficacy of 100 mg DCS as an adjunctive strategy to iTBS to reduce OCD-related cognitive impairment and/or improve cognition in OCD was tested in a randomized double-blind 4-week study described in the Example above.

Participants completed the Wisconsin Card Sorting Task at baseline and after 4 weeks of treatment. The preplanned focus was on the percent perseverative errors as an index of executive function, cognitive flexibility and cognitive control.

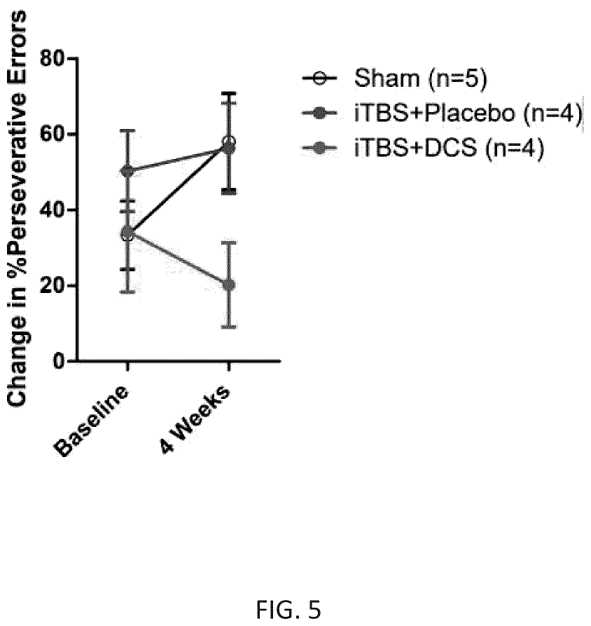

Results: Referring to FIG. 5, the group treated with iTBS+DCS had a decrease in the percent perseverative errors and that the slope of this relationship was significantly different than changes in percent perseverative errors in the iTBS+Placebo and Sham groups.

Conclusions: Adjunctive DCS with iTBS improves executive function and cognitive control in OCD relative to iTBS+Placebo and Sham-iTBS.

### REFERENCES

1. Ruscio A M, Stein D J, Chiu W T, Kessler R C. The epidemiology of obsessive-compulsive disorder in the National Comorbidity Survey Replication. Mol Psychiatry 2010; 15(1): 53-63.
2. Stein M B, Forde D R, Anderson G, Walker J R. Obsessive-compulsive disorder in the community: an epidemiologic survey with clinical reappraisal. Am J Psychiatry 1997; 154(8): 1120-1126.
3. Global burden of 369 diseases and injuries in 204 countries and territories, 1990-2019: a systematic analysis for the Global Burden of Disease Study 2019. Lancet 2020; 396(10258): 1204-1222.
4. DuPont R L, Rice D P, Shiraki S, Rowland C R. Economic costs of obsessive-compulsive disorder. Med Interface 1995; 8(4): 102-109.
5. Soomro G M, Altman D, Rajagopal S, Oakley-Browne M. Selective serotonin re-uptake inhibitors (SSRIs) versus placebo for obsessive compulsive disorder (OCD). Cochrane Database Syst Rev 2008; 2008(1): CD001765.
6. Kamath P, Reddy Y C, Kandavel T. Suicidal behavior in obsessive-compulsive disorder. J Clin Psychiatry 2007; 68(11): 1741-1750.
7. Brakoulias V, Starcevic V, Belloch A, Brown C, Ferrao Y A, Fontenelle L F et al. Comorbidity, age of onset and suicidality in obsessive-compulsive disorder (OCD): An international collaboration. Compr Psychiatry 2017; 76: 79-86.
8. Torres A R, Ramos-Cerqueira A T, Ferrão Y A, Fontenelle L F, do Rosário M C, Miguel E C. Suicidality in obsessive-compulsive disorder: prevalence and relation to symptom dimensions and comorbid conditions. J Clin Psychiatry 2011; 72(1): 17-26; quiz 119-120.
9. Alonso P, Segalàs C, Real E, Pertusa A, Labad J, Jiménez-Murcia S et al. Suicide in patients treated for obsessive-compulsive disorder: a prospective follow-up study. J Affect Disord 2010; 124(3): 300-308.
10. Purcell R, Maruff P, Kyrios M, Pantelis C. Neuropsychological deficits in obsessive-compulsive disorder: a comparison with unipolar depression, panic disorder, and normal controls. Arch Gen Psychiatry 1998; 55(5): 415-423.
11. Shin N Y, Lee T Y, Kim E, Kwon J S. Cognitive functioning in obsessive-compulsive disorder: a meta-analysis. Psychol Med 2014; 44(6): 1121-1130.
12. Chamberlain S R, Solly J E, Hook R W, Vaghi M M, Robbins T W. Cognitive Inflexibility in OCD and Related Disorders. Curr Top Behav Neurosci 2021; 49: 125-145.
13. D'Alcante C C, Diniz J B, Fossaluza V, Batistuzzo M C, Lopes A C, Shavitt R G et al. Neuropsychological predictors of response to randomized treatment in obsessive-compulsive disorder. Prog Neuropsychopharmacol Biol Psychiatry 2012; 39(2): 310-317.
14. Bannon S, Gonsalvez C J, Croft R J, Boyce P M. Executive functions in obsessive-compulsive disorder: state or trait deficits? Aust N Z J Psychiatry 2006; 40(11-12): 1031-1038.
15. Carmi L, Tendler A, Bystritsky A, Hollander E, Blumberger D M, Daskalakis J et al. Efficacy and Safety of Deep Transcranial Magnetic Stimulation for Obsessive-Compulsive Disorder: A Prospective Multicenter Randomized Double-Blind Placebo-Controlled Trial. Am J Psychiatry 2019; 176(11): 931-938.
16. Huang Y Z, Chen R S, Rothwell J C, Wen H Y. The after-effect of human theta burst stimulation is NMDA receptor dependent. Clin Neurophysiol 2007; 118(5): 1028-1032.
17. Andersson E, Hedman E, Enander J, Radu Djurfeldt D, Ljótsson B, Cervenka S et al. D-Cycloserine vs Placebo as Adjunct to Cognitive Behavioral Therapy for Obsessive-Compulsive Disorder and Interaction With Antidepressants: A Randomized Clinical Trial. JAMA Psychiatry 2015; 72(7): 659-667.
18. Mataix-Cols D, Turner C, Monzani B, Isomura K, Murphy C, Krebs G et al. Cognitive-behavioural therapy with post-session D-cycloserine augmentation for paediatric obsessive-compulsive disorder: pilot randomised controlled trial. Br J Psychiatry 2014; 204(1): 77-78.
19. Storch E A, Merlo L J, Bengtson M, Murphy T K, Lewis M H, Yang M C et al. D-cycloserine does not enhance exposure-response prevention therapy in obsessive-compulsive disorder. Int Clin Psychopharmacol 2007; 22(4): 230-237.
20. Storch E A, Wilhelm S, Sprich S, Henin A, Micco J, Small B J et al. Efficacy of Augmentation of Cognitive Behavior Therapy With Weight-Adjusted d-Cycloserine vs Placebo in Pediatric Obsessive-Compulsive Disorder: A Randomized Clinical Trial. JAMA Psychiatry 2016; 73(8): 779-788.
21. Kuhn M, Mainberger F, Feige B, Maier J G, Wirminghaus M, Limbach L et al. State-Dependent Partial Occlusion of Cortical LTP-Like Plasticity in Major Depression. Neuropsychopharmacology 2016; 41(6): 1521-1529.
22. Player M J, Taylor J L, Weickert C S, Alonzo A, Sachdev P, Martin D et al. Neuroplasticity in depressed individuals compared with healthy controls. Neuropsychopharmacology 2013; 38(11): 2101-2108.
23. Vignaud P, Damasceno C, Poulet E, Brunelin J. Impaired Modulation of Corticospinal Excitability in Drug-Free Patients With Major Depressive Disorder: A Theta-Burst Stimulation Study. Front Hum Neurosci 2019; 13: 72.
24. Greenberg B D, Ziemann U, Corá-Locatelli G, Harmon A, Murphy D L, Keel J C et al. Altered cortical excitability in obsessive-compulsive disorder. Neurology 2000; 54(1): 142-147.
25. Greenberg B D, Ziemann U, Harmon A, Murphy D L, Wassermann E M. Decreased neuronal inhibition in cerebral cortex in obsessive-compulsive disorder on transcranial magnetic stimulation. Lancet 1998; 352(9131): 881-882.
26. Kang J I, Kim D Y, Lee Cl, Kim C H, Kim S J. Changes of motor cortical excitability and response inhibition in patients with obsessive-compulsive disorder. J Psychiatry Neurosci 2019; 44(4): 261-268.
27. Khedr E M, Elbeh K A, Elserogy Y, Khalifa H E, Ahmed M A, Hafez M H et al. Motor cortical excitability in obsessive-compulsive disorder: Transcranial magnetic stimulation study. Neurophysiol Clin 2016; 46(2): 135-143.
28. Radhu N, de Jesus D R, Ravindran L N, Zanjani A, Fitzgerald P B, Daskalakis Z J. A meta-analysis of cortical inhibition and excitability using transcranial magnetic stimulation in psychiatric disorders. Clin Neurophysiol 2013; 124(7): 1309-1320.
29. Richter M A, de Jesus D R, Hoppenbrouwers S, Daigle M, Deluce J, Ravindran L N et al. Evidence for cortical inhibitory and excitatory dysfunction in obsessive compulsive disorder. Neuropsychopharmacology 2012; 37(5): 1144-1151.
30. Roth Y, Tendler A, Arikan M K, Vidrine R, Kent D, Muir O et al. Real-world efficacy of deep TMS for obsessive-compulsive disorder: Post-marketing data collected from twenty-two clinical sites. J Psychiatr Res 2020; 4(20): 31065-31067.
31. Brown J C, DeVries W H, Korte J E, Sahlem G L, Bonilha L, Short E B et al. NMDA receptor partial agonist, d-cycloserine, enhances 10 Hz rTMS-induced motor plasticity, suggesting long-term potentiation (LTP) as underlying mechanism. Brain Stimul 2020; 13(3): 530-532.
32. Traynelis S F, Wollmuth L P, McBain C J, Menniti F S, Vance K M, Ogden K K et al. Glutamate receptor ion channels: structure, regulation, and function. Pharmacol Rev 2010; 62(3): 405-496.
33. Popoli M, Yan Z, McEwen B S, Sanacora G. The stressed synapse: the impact of stress and glucocorticoids on glutamate transmission. Nat Rev Neurosci 2011; 13(1): 22-37.
34. Yuen E Y, Liu W, Karatsoreos I N, Feng J, McEwen B S, Yan Z. Acute stress enhances glutamatergic transmission in prefrontal cortex and facilitates working memory. Proc Natl Acad Sci USA 2009; 106(33): 14075-14079.
35. Yuen E Y, Wei J, Liu W, Zhong P, Li X, Yan Z. Repeated stress causes cognitive impairment by suppressing glutamate receptor expression and function in prefrontal cortex. Neuron 2012; 73(5): 962-977.
36. Dravid S M, Burger P B, Prakash A, Geballe M T, Yadav R, Le P et al. Structural determinants of D-cycloserine efficacy at the NR1/NR2C NMDA receptors. J Neurosci 2010; 30(7): 2741-2754.
37. Sheinin A, Shavit S, Benveniste M. Subunit specificity and mechanism of action of NMDA partial agonist D-cycloserine. Neuropharmacology 2001; 41(2): 151-158.
38. Li C T, Chen M H, Juan C H, Huang H H, Chen L F, Hsieh J C et al. Efficacy of prefrontal theta-burst stimulation in refractory depression: a randomized sham-controlled study. Brain 2014; 137(Pt 7): 2088-2098.
39. van Berckel B N, Evenblij C N, van Loon B J, Maas M F, van der Geld M A, Wynne H J et al. D-cycloserine increases positive symptoms in chronic schizophrenic patients when administered in addition to antipsychotics: a double-blind, parallel, placebo-controlled study. Neuropsychopharmacology 1999; 21(2): 203-210.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

We claim:

1. A method of treating Obsessive Compulsive Disorder (OCD) comprising administering adjunctive D-Cycloserine or a pharmaceutically acceptable ester of D-Cycloserine, or an alkylated D-Cycloserine, or a pharmaceutically acceptable precursor of D-Cycloserine to augment transcranial magnetic stimulation (TMS) therapy for OCD, optionally intermittent or continuous theta-burst stimulation, high- or low-frequency stimulation, and combinations thereof, to improve and/or alleviate and/or reduce frequency of one or more symptoms of OCD.

2. A method of treating OCD in a patient and/or improving and/or alleviating and/or reducing the frequency of one or more symptom of OCD, the method comprising: administering to the patient a low dose of D-Cycloserine, optionally 1-250 mg per patient; and subjecting the patient to transcranial magnetic stimulation, optionally intermittent or continuous theta-burst stimulation, high- or low-frequency stimulation, and combinations thereof.

3. (canceled)

4. The method of claim 2, wherein the low dose is a dose of D-cycloserine sufficient to achieve a plasma concentration of D-cycloserine of 1-30 μg/mL.

5. The method of claim 2, wherein the patient is subjected to intermittent or continuous theta-burst stimulation, high- or low-frequency stimulation, and combinations thereof, when plasma concentration of D-cycloserine is between 1-30 μg/mL.

6. The method of claim 1, wherein D-Cycloserine is administrated at a dose to achieve maximal plasma levels of 1-30 μg/mL within six hours of the transcranial magnetic stimulation.

7. A method of improving and/or reversing and/or partially reversing cognitive impairment in OCD, including executive function and cognitive control, the method comprising: administering to the patient a low dose of D-Cycloserine and subjecting the patient to transcranial magnetic stimulation, optionally intermittent or continuous theta-burst stimulation, high- or low-frequency stimulation, and combinations thereof.

8. The method of claim 7, wherein the low dose is a dose of D-cycloserine sufficient to achieve a plasma concentration of D-cycloserine of 1-30 μg/mL.

9. The method of claim 7, wherein the patient is subjected to intermittent or continuous theta-burst stimulation, high- or low-frequency stimulation, and combinations thereof, when plasma concentration of D-cycloserine is between 1-30 μg/mL.
